(19) United States
(12) Patent Application Publication
(10) Pub. No.: US 2006/0229723 A1
(43) Pub. Date: Oct. 12, 2006

(54) Intervertebral fusion device and method

(75) Inventor: James E. Van Hoeck, Cordova, TN (US)

Correspondence Address: HAYNES AND BOONE, LLP, 901 MAIN STREET, SUITE 3100, DALLAS, TX, 75202, US

(73) Assignee: SDGI Holdings, Inc., Wilmington, DE (US)

(21) Appl. No.: 11/101,685

(22) Filed: Apr. 8, 2005

(51) Int. Cl. A61F 2/44

(52) U.S. Cl. 623/17.11

(57) ABSTRACT

A intervertebral fusion device and method according to which an artificial disc is removed from an intervertebral space between adjacent vertebrae and an intervertebral fusion device is inserted in the intervertebral space to fuse the adjacent vertebrae.

### BACKGROUND

The present disclosure relates in general to spinal fusion and in particular to intervertebral fusion devices.

Spinal discs that extend between the endplates of adjacent vertebrae in a spinal column of the human body provide critical support between the adjacent vertebrae. These discs can rupture, degenerate and/or protrude by injury, degradation, disease or the like to such a degree that the intervertebral space between adjacent vertebrae collapses as the disc loses at least a part of its support function, which can cause impingement of the nerve roots and severe pain. In some cases, surgical correction may be required.

Typically, the surgical correction includes the removal of the spinal disc from between the adjacent vertebrae, and, in order to preserve the intervertebral disc space for proper spinal-column function, a prosthetic device is sometimes inserted in the intervertebral space between the adjacent vertebrae. In this context, the prosthetic device may be referred to as an artificial disc, an intervertebral prosthetic joint or a prosthetic implant, among other labels.

In some cases, the inserted artificial disc may not function properly due to a wide variety of reasons such as, for example, failure of or damage to the artificial disc, poor tissue healing, the deterioration of the function and/or shape of the spinal column after the surgical correction, and/or other patient-related factors. In response, revision surgery, that is, another surgical correction, may be required in which the artificial disc is removed from the intervertebral space between the adjacent vertebrae. During revision surgery, another artificial disc may be inserted in the intervertebral space between the adjacent vertebrae, or the adjacent vertebrae may instead be fused or joined together using a variety of fusion techniques such as, for example, inserting an implant, spacer or cage in the intervertebral space between the adjacent vertebrae so that the implant or spacer engages the adjacent vertebrae. In the case of the latter, bone in-growth into the intervertebral space and/or the implant, spacer or cage may occur, thereby fusing or joining the adjacent vertebrae.

However, one or more issues may arise if the adjacent vertebrae are fused by inserting an implant, spacer or cage in the intervertebral space between the adjacent vertebrae. For example, appreciable amounts of bone or other material may have to be removed from one or more of the adjacent vertebrae for one or more reasons such as, for example, to fit the implant, spacer or cage in the intervertebral space between the adjacent vertebrae. Also, the engagements between the implant, spacer or cage and each of the adjacent vertebrae may not effectively promote new bone growth due to the lack of stability and/or alignment across the interfaces between the implant, spacer or cage and each of the adjacent vertebrae. This lack of stability and/or alignment may stem from the contours defined by the endplates of the adjacent vertebrae, and other features of the endplates, which may be formed and/or defined by the insertion and subsequent removal of the artificial disc, and which may not be effectively physically compatible with the implant, spacer or cage.

### SUMMARY

An intervertebral fusion device adapted to be inserted in an intervertebral space between adjacent first and second vertebrae for fusing the first and second vertebrae is provided that includes a first portion adapted to engage the first vertebra, the first portion defining a first geometry wherein at least a portion of the first geometry generally corresponds to at least one portion of a geometry defined by an artificial disc.

A method for fusing adjacent vertebrae is provided that includes inserting an intervertebral fusion device in an intervertebral space between the adjacent vertebrae and engaging a first portion of the intervertebral fusion device with one of the adjacent vertebrae wherein the first portion defines a first geometry that generally corresponds to at least one portion of a geometry defined by an artificial disc.

An intervertebral fusion device is provided that is adapted to be inserted in an intervertebral space between adjacent vertebrae to fuse the adjacent vertebrae and that defines a geometry that generally corresponds to the geometry of an artificial disc adapted to be inserted in the intervertebral space, wherein the device is adapted to be inserted in the intervertebral space and engaged with the adjacent vertebrae after the artificial disc is inserted and removed from the intervertebral space, and wherein the general correspondence between the geometry of the device and the geometry of the artificial disc facilitates the engagements between the device and each of the adjacent vertebrae.

### BRIEF DESCRIPTION OF THE DRAWINGS

FIG. 1 is a perspective view of an intervertebral fusion device according to an embodiment.

FIG. 2 is a perspective view of an artificial disc.

FIG. 3 is an elevational view of the artificial disc of FIG. 2 being removed from an intervertebral space between adjacent vertebrae.

FIG. 4 is a sectional view depicting the intervertebral fusion device of FIG. 1 inserted in the intervertebral space between the adjacent vertebrae of FIG. 2.

FIG. 5 is a perspective view of an intervertebral fusion device according to another embodiment.

FIG. 6 is an anterior elevational view of the intervertebral fusion device of FIG. 5 inserted in an intervertebral space between adjacent vertebrae.

FIG. 7 is a perspective view of an intervertebral fusion device according to another embodiment.

FIG. 8 is a perspective view of an intervertebral fusion device according to another embodiment inserted in an intervertebral space between adjacent vertebrae.

### DETAILED DESCRIPTION

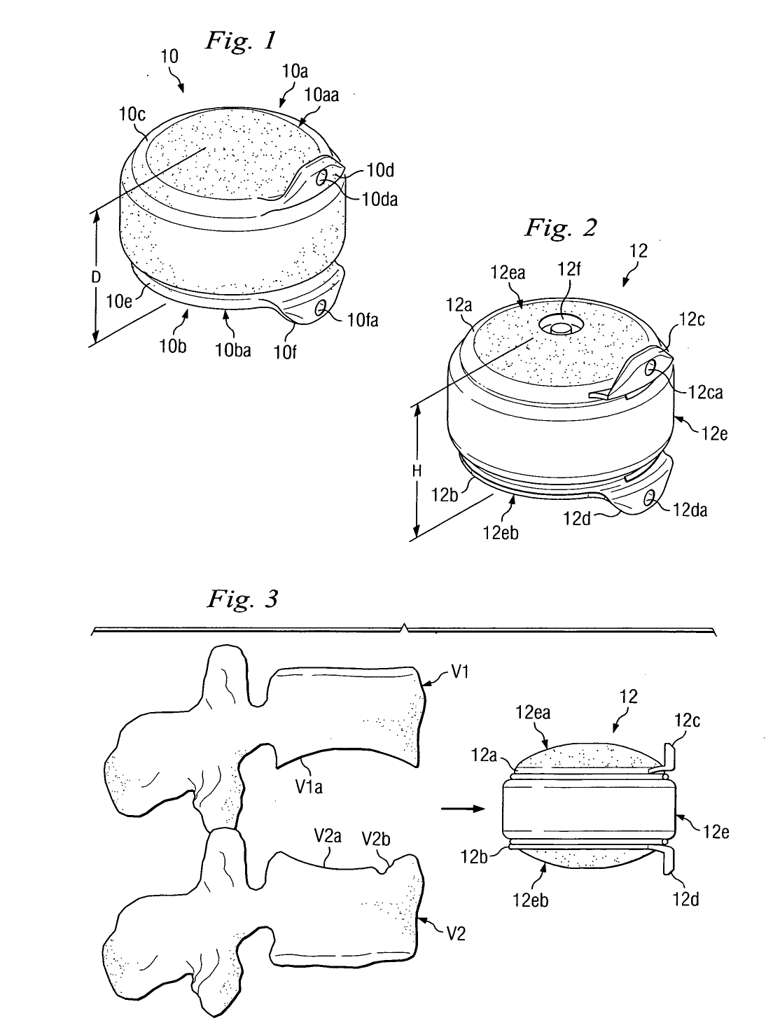

Referring to FIG. 1, an intervertebral fusion device is generally referred to by the reference numeral 10 and includes an upper portion 10a and a lower portion 10b. The device 10 is a solid unitary or monolithic piece that generally corresponds to, or matches, an artificial disc 12 that is shown in FIG. 2. It is understood that the artificial disc 12 is an articulating joint configured for insertion between adjacent vertebrae in a human spine to maintain or restore motion by providing relative bending, translational and/or rotational motion between the adjacent vertebrae. It is further understood that the artificial disc 12 may also be referred to as a prosthetic joint, prosthetic implant or disc prostheses, among other labels.

As shown in FIG. 2, the disc 12 is similar to a BRYAN® artificial disc and includes shells 12a and 12b, flanges 12c and 12d extending from the shells 12a and 12b, respectively, holes 12ca and 12da formed through the flanges 12c and 12d, respectively, and other components, resulting in an overall size and shape that defines a geometry 12e having an upper portion 12ea and a lower portion 12eb. It is understood that the geometry 12e includes the size and the shape of the overall external surface of the disc 12, including the sizes and shapes of the external surfaces of the shells 12a and 12b, the flanges 12c and 12d, and the other components of the disc 12, all of which are described in detail in U.S. Patent Publication No. 2003/0135277 (U.S. Ser. No. 10/303,569), the disclosure of which is incorporated by reference. It is understood that, when the disc 12 is inserted between adjacent vertebrae, at least a portion of the upper portion 12ea of the geometry 12e is adapted to engage one of the vertebrae and at least a portion of the lower portion 12eb of the geometry 12e is adapted to engage the other of the adjacent vertebrae.

The upper portion 10a of the device 10 defines a geometry 10a that generally corresponds to, or matches, the upper portion 12ea of the geometry 12e of the disc 12 and, as such, includes a shell-shaped portion 10c, a flange 10d extending from the shell-shaped portion 10c, and a hole 10aformed through the flange 10d. Similarly, the lower portion 10b of the device 10 defines a geometry 10a that generally corresponds to, or matches, the lower portion 12eb of the geometry 12e of the disc 12 and, as such, includes a shell-shaped portion 10e, a flange 10f extending from the shell-shaped portion 10e, and a hole 10fa formed through the flange 10f. Moreover, the portions 10a and 10b of the device 10 define a dimension D that generally corresponds to an effective height H of the disc 12.

It is understood that the geometries 10aa and 10ba of the device 10 may vary or be modified to some degree from the portions 12ea and 12eb, respectively, of the geometry 12e of the disc 12, while still maintaining a general correspondence therebetween, at least with respect to the overall size and shape and the relatively large features of the portions 12ea and 12eb of the geometry 12e. For example, although the disc 12 includes an access port 12fthat defines one or more external surfaces of the portion 12ea of the geometry 12e of the disc 12, the external surfaces defined by the access port 12f are not reflected in the geometry 10aa of the device 10. For another example, although not depicted in FIG. 1, the flanges 10d and 10f may be removed from the device 10.

The device 10 is at least partially composed of an osteogenic material, that is, a material that has the ability to promote, enhance and/or accelerate the growth of new bone tissue by one or more mechanisms such as, for example, osteogenesis, osteoconduction and/or osteoinduction. Examples of osteogenic materials include, but are not limited to, all types of bone and synthetic bone materials, including various types of allograft, autograft, allogenic and/or xenogenic materials, and/or any combination thereof.

In operation, and referring to FIG. 3 with continuing reference to FIGS. 1 and 2, the artificial disc 12 is removed in a conventional manner during revision surgery from between adjacent vertebrae V1 and V2, as indicated by the direction of the arrow in FIG. 3. Due to the removal of the disc 12, contours generally corresponding to the geometries 12ea and 12eb of the disc 12 are defined by endplates V1a and V2a, respectively, of the vertebrae V1 and V2, respectively. Moreover, a cavity V2b is formed in the endplate V2a due to material loss from bone in-growth into the disc 12. That is, as the disc 12 is removed, bone or other natural material, that is connected to the disc 12 due to in-growth, is also removed from the vertebra V2, resulting in the formation of the cavity V2b. It is understood that, due to the removal of the disc 12, additional bone and/or other material loss may occur in the vicinity of the vertebrae V1 and V2, including other locations on the vertebrae V1 and/or V2.

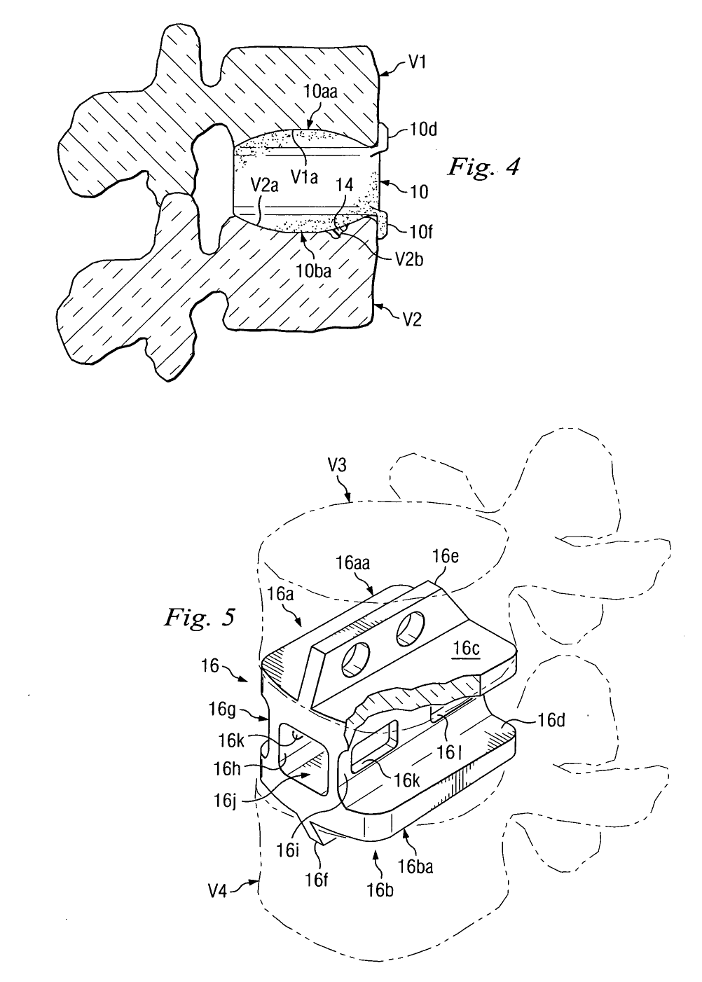

Referring to FIG. 4, with continuing reference to FIGS. 1 through 3, the device 10 is inserted in the intervertebral space between the vertebrae V1 and V2. At least a portion of the upper portion 10a of the device 10 engages the endplate V1a, and at least a portion of the lower portion 10b of the device 10 engages the endplate V2a. The dimension D defined by the portions 10a and 10b of the device 10 is substantially equal to the height of the intervertebral space between the vertebrae V1 and V2.

Since the geometry 10aa of the upper portion 10a of the device 10 generally corresponds to the portion 12ea of the geometry 12e of the disc 12, the engagement between the upper portion 10a of the device 10 and the vertebra V1 is facilitated, thereby assisting to minimize the need for any material removal from the endplate V1a and/or other areas of the vertebra V1. Likewise, since the geometry 10ba of the lower portion 10b of the device 10 generally corresponds to the portion 12eb of the geometry 12e of the disc 12, the engagement between the lower portion 10b of the device 10 and the vertebra V2 is facilitated, thereby assisting to minimize the need for any material removal from the endplate V2a and/or other areas of the vertebra V2.

Before, during and/or after the insertion of the device 10 into the intervertebral space between the vertebrae V1 and V2, filler material 14 may be disposed in the cavity V2b. The filler material 14 may be composed of any type of material that has the ability to promote, enhance and/or accelerate the growth of new bone tissue by one or more mechanisms such as, for example, osteogenesis, osteoconduction and/or osteoinduction, including the types of materials identified above in connection with the device 10, or any combination thereof. Moreover, the filler material 14 may be in the form of, for example, allograft chips, bone marrow, a calcium phosphate ceramic, a demineralized bone matrix putty or gel and/or any combination thereof. It is understood that the filler material 14 may be injected into the cavity V2b if the filler material 14 is in the form of, for example, bone marrow, a bone matrix gel or a calcium phosphate cement which later hardens into a calcium phosphate ceramic within the cavity V2b. It is further understood that the filler material 14 may be disposed in openings formed due to additional bone and/or other material loss in the vicinity of the vertebrae V1 and V2, including openings formed at other locations on the vertebrae V1 and/or V2.

After the insertion of the device 10 between the vertebrae V1 and V2 has been completed, the device 10 promotes the fusion or joining together of the vertebrae V1 and V2. During this fusion, the above-described structural and material properties of the device 10 allow and promote the growth of new bone material between the vertebrae V1 and V2 so that the vertebrae V1 and V2 biologically grow together and form a solid mass, thereby stabilizing the spine of which the vertebrae V1 and V2 are a part. It is understood that bone in-growth may occur into any interconnected pores of the material of the device 10, from any natural source in the vicinity of the vertebrae V1 and V2, including the vertebrae V1 and V2.

Due to its above-described material properties, it is understood that the filler material 14 in the cavity V2b also promotes bone growth, serving as an adjunct to the fusion promotion of the device 10. It is further understood that the device 10 has sufficient rigidity and structural integrity to substantially maintain the dimension D, and therefore the height of the intervertebral space between the vertebrae V1 and V2, and to withstand any internal or external forces applied to the spinal column of which the vertebrae V1 and V2 are a part.

The engagements between the device 10 and the vertebrae V1 and V2, which are facilitated by the general correspondence of the geometries 10aa and 10ba with the contours of the endplates V1a and V2a, respectively, assist in maintaining alignment and stability across the interface between the vertebra V1 and the device 10, and across the interface between the device 10 and the vertebra V2, thereby further promoting new bone growth and the fusion of the vertebrae V1 and V2.

It is understood that, to provide further alignment and/or stabilization during the fusion of the vertebrae V1 and V2, hardware may be implemented in the vicinity of the device 10. For example, nonresorbable or resorbable fasteners or anchors may be inserted through the holes 10da and 10fa formed through the flanges 10d and 10f, respectively, so that the anchors extend into the vertebra V1 and V2, respectively, further securing the device 10 to the vertebra V1 and V2 and assisting in maintaining alignment and stability. It is further understood that one or more additional flanges, rods, plates and/or other components of supplemental fixation systems may extend from the device 10 and that additional fasteners may extend through the same and/or the device 10 in order to provide increased alignment and stability, and/or one or more other supplemental fixation systems and/or components thereof may engage the vertebrae V1 and/or V2 and/or extend or be disposed in the vicinity thereof in order to provide increased alignment and stability. One example of a flange configuration that may be added to the device 10 is described in detail in U.S. Pat. No. 6,562,073, the disclosure of which is incorporated by reference.

It is understood that, instead of or in addition to being at least partially composed of one or more osteogenic materials, the device 10 may be composed of any type of solid or semi-solid material, regardless of whether the solid or semi-solid material promotes, enhances and/or accelerates bone growth. Moreover, it is understood that the device 10 may be composed out of any type of composite material and/or out of any combination of any of the above-identified material types, among other materials and material types. It is further understood that the device 10 may be coated with one or more of the foregoing types of osteogenic materials. It is further understood that the material of which the device 10 is composed may have specific predetermined properties and/or may undergo one or more processes known to those skilled in the art such as, for example, the processes described in U.S. Pat. No. 6,696,073, the disclosure of which is incorporated by reference.

To manufacture the device 10 to generally correspond to the disc 12, the osteogenic material described above may be subjected to one or more of a wide variety of conventional manufacturing techniques and processes such as, for example, precision machining. Other conventional manufacturing techniques and processes that may be employed include, but are not limited to, various types of extruding methods, various types of molding methods including compression molding, various types of casting methods including solvent or solution casting, vacuum-forming methods, sintering methods and/or any combination and/or variation thereof.

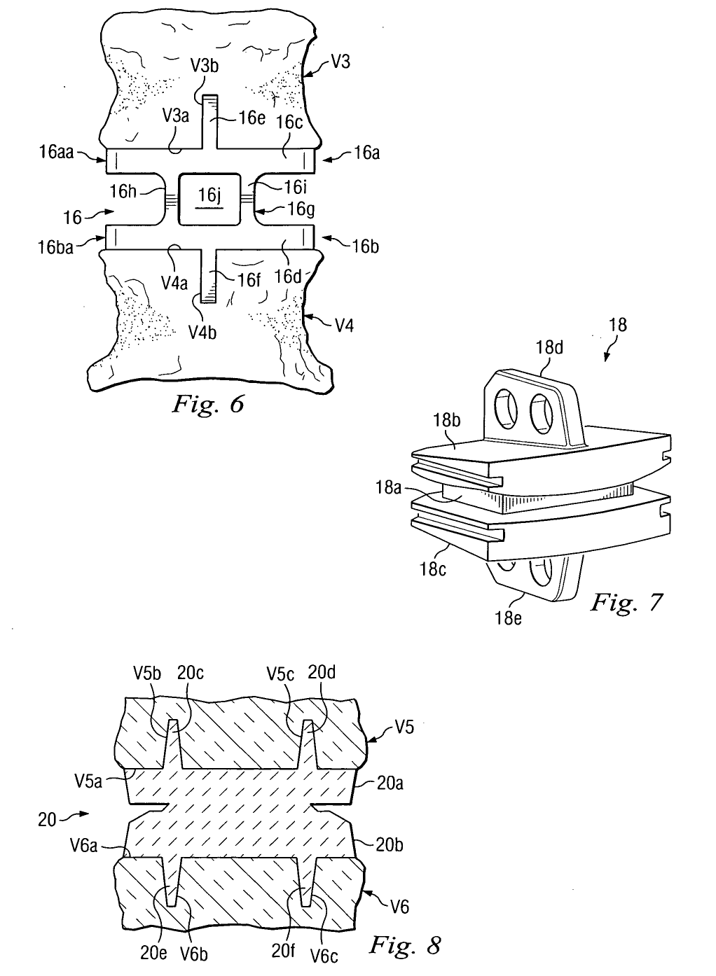

Referring to FIGS. 5 and 6, another embodiment of an intervertebral fusion device is generally referred to by the reference numeral 16 and includes an upper portion 16a and a lower portion 16b. The portions 16a and 16b of the device 16 define geometries 16aa and 16ba, respectively, that generally correspond to, or match, one or more features of the MAVERICKS artificial disc and/or one or more features described in detail in U.S. Pat. No. 6,740,118, the disclosure of which is incorporated by reference. As such, the portions 16a and 16b include support-plate-shaped portions 16c and 16d, respectively, and protrusion or keel-shaped portions 16e and 16f, respectively, extending therefrom.

A cage portion 16g includes a pair of parallel-spaced walls 16h and 16i extending between the portions 16a and 16b. A chamber 16j is defined by the walls 16h and 16i and the portions 16a and 16b, and is adapted to receive an osteogenic material such as, for example, autograft bone, a bone substitute material and/or any of the materials identified above in connection with the device 10 and/or the filler material 14 in the embodiment of FIGS. 1 through 4, and/or any combination or variation thereof. Apertures 16k and 161 transversely extend through the walls 16h and 16i (the extension of the aperture 161 through the wall 16h is not shown).

The device 16 is at least partially composed of an osteogenic material such as, for example, one or more of the types of osteogenic materials described above in connection with the device 10 in the embodiment of FIGS. 1 through 4.

It is understood that the device 16 is adapted to be inserted in an intervertebral space between adjacent vertebrae V3 and V4. Prior to the insertion of the device 16 between the vertebrae V3 and V4, an artificial disc in the form of a MAVERICK® artificial disc, or in the form of an artificial disc embodiment described in detail in U.S. Pat. No. 6,740,118, the disclosure of which is incorporated by reference, is removed during revision surgery from between the adjacent vertebrae V3 and V4 and, as a result, contours are defined by the endplates V3a and V4a of the vertebrae V3 and V4, respectively. As a further result, channels V3b and V4b are defined in the vertebrae V3 and V4, respectively.

Thereafter, the device 16 is inserted in the intervertebral space between the adjacent vertebrae V3 and V4. At least a portion of the plate-shaped portion 16c engages the endplate V3a and the keel-shaped portion 16e extends into the channel V3b. Similarly, at least a portion of the plate-shaped portion 16d engages the endplate V4a and the keel-shaped portion 16f extends into the channel V4b. In view of the foregoing engagements, it is understood that the general correspondence between the geometries 16aa and 16ba, and the features of the MAVERICK® artificial disc and/or one or more features described in detail in U.S. Pat. No. 6,740,118, the disclosure of which is incorporated by reference, facilitates the engagement between the upper portion 16a and the endplate V3a, and the engagement between the lower portion 16b and the endplate V4a, thereby assisting to minimize the need for any material removal from the endplates V3a and V4a and/or other areas of the vertebrae V3 and V4.

Before, during and/or after the insertion of the device 16 in the intervertebral space between the vertebrae V3 and V4, the chamber 16j receives an osteogenic material such as, for example, autograft bone, a bone substitute material and/or any of the materials identified above in connection with the device 10 and/or the filler material 14 in the embodiment of FIGS. 1 through 4, and/or any combination or variation thereof, It is understood that additional osteogenic material may be disposed in other locations between the plate-shaped portions 16c and 16d.

After the insertion of the device 16 in the intervertebral space between the vertebrae V3 and V4, the device 16 promotes the fusion or joining together of the vertebrae V3 and V4 so that the vertebrae V3 and V4 biologically grow together and form a solid mass, thereby stabilizing the spine of which the vertebrae V3 and V4 are a part. The osteogenic material disposed in the chamber 16j further promotes a solid fusion across the vertebrae V3 and V4.

The engagements between the plate-shaped portion 16c and the endplate V3a, and between the keel-shaped portion 16e and the channel V3b, assist in maintaining alignment and stability across the interface between the vertebra V3 and the device 16, thereby further promoting new bone growth and the fusion of the vertebrae V3 and V4. Likewise, the engagements between the plate-shaped portion 16d and the endplate V4a, and between the keel-shaped portion 16f and the channel V4b, assist in maintaining alignment and stability across the interface between the device 16 and the vertebra V4, thereby further promoting new bone growth and the fusion of the vertebrae V3 and V4.

Apertures 16k and 161 of the cage portion 16g provide a passageway for vacscularization to occur between the osteogenic material disposed in the chamber 16j and the natural human tissue in the vicinity of the vertebrae V3 and V4. Bone in-growth may also occur through the apertures 16k and 161, further assisting in the fusion of the vertebrae V3 and V4.

It is understood that, instead of or in addition to being at least partially composed of an osteogenic material, the device 16 may be composed of any type of biocompatible or inert material such as, for example, medical grade stainless steel or titanium, a biocompatible porous material such as a porous tantalum composite and/or any other type of material described in U.S. Pat. No. 6,613,091, the disclosure of which is incorporated by reference. Also, it is understood that the cage portion 16g may be in the form of a wide variety of fusion-cage configurations such as, for example, the fusion-cage configurations described in U.S. Pat. Pub. No. 2005/0060034 (U.S. Ser. No. 10/662,928), the disclosure of which is incorporated by reference, or, in the alternative, the cage portion 16g may be in the form of solid portion extending between the plate-shaped portions 16c and 16d. Moreover, it is understood that the device 16 may be in the form of a solid unitary or monolithic piece, or may be in the form of assembly in which, for example, the plate-shaped portions 16c and 16d are fastened to the cage portion 16g.

It is further understood that one or more slots, holes and/or other through-openings may be formed through the plate-shaped portions 16c and 16d to promote bone in-growth therethrough from the vertebrae V3 and V4, respectively, and to the osteogenic material disposed in the chamber 16j of the cage portion 16g. It is understood that the osteogenic material disposed in the chamber 16j may extend through these slots, holes and/or through-openings and directly engage the vertebrae V3 and V4. Moreover, it is understood that stabilizing hardware such as, for example, plates, flanges, rods and/or fasteners, may be added to the device 16, and/or to the vertebrae V3 and V4 or the vicinity thereof, to provide increased alignment and stability between the device 16 and the vertebrae V3 and V4, including the hardware described above in connection with the device 10 in the embodiment of FIGS. 1 through 4.

Referring to FIG. 7, another embodiment of an intervertebral fusion device is generally referred to by the reference numeral 18. The device 18 is a solid unitary or monolithic piece having a geometry that generally corresponds to, or matches, the geometry of a MAVERICK® artificial disc or one or of the artificial disc embodiments described in detail in U.S. Pat. No. 6,740,118, the disclosure of which is incorporated by reference. A solid middle portion 18a of the device 18 extends between plate-shaped portions 18b and 18c, and protrusion or keel-shaped portions 18d and 18e extend from the plate-shaped portions 18b and 18c, respectively. The device 18 is at least partially composed of an osteogenic material such as, for example, any one of the osteogenic materials described above in connection with the device 10 in the embodiment of FIGS. 1 through 4.

The operation of the device 18 is substantially similar to the operation of the device 10 in the embodiment of FIGS. 1 through 4 and therefore will not be described in detail. It is understood that, prior to the insertion of the device 18 in an intervertebral space between adjacent vertebrae, an artificial disc, in the form of a MAVERICK® artificial disc or one of the artificial disc embodiments described in detail in U.S. Pat. No. 6,740,118, the disclosure of which is incorporated by reference, is removed during revision surgery from the intervertebral space between the adjacent vertebrae. Due to the above-described geometry of the device 18, the engagements between the device 18 and the vertebrae V3 and V4 are facilitated, thereby assisting to minimize the need for material removal from the vertebrae V3 and/or V4.

Referring to FIG. 8, another embodiment of a intervertebral fusion device is generally referred to by the reference numeral 20 and is adapted to be inserted between adjacent vertebrae V5 and V6. The device 20 is a solid unitary or monolithic piece having a geometry that generally corresponds to, or matches, the geometry of a PRODISC® artificial disc or one of the artificial disc embodiments disclosed in U.S. Pat. No. 5,314,477, the disclosure of which is incorporated by reference, and, as such, includes an upper portion 20a and a lower portion 20b. Protrusion or anchoring-flap portions 20c and 20d extend from the upper portion 20a, and protrusion or anchoring-flap portions 20e and 20f extend from the lower portion 20b. The device 20 is at least partially composed of an osteogenic material such as, for example, any one of the osteogenic materials described above in connection with the device 10 in the embodiment of FIGS. 1 through 4. It is understood that the geometry of the device 20 may vary or be modified to some degree from the geometry of a PRODISC® artificial disc or one of the artificial disc embodiments disclosed in U.S. Pat. No. 5,314,477, the disclosure of which is incorporated by reference, while still maintaining a general correspondence therebetween. For example, the upper portion 20a and the lower portion 20b may each extend in a generally continuous vertical direction towards the other, resulting in the device 20 having a generally rectangular cross-section between the anchoring-flap portions 20c and 20d and the anchoring-flap portions 20e and 20f.

The operation of the device 20 is substantially similar to the operation of the device 10 in the embodiment of FIGS. 1 through 4 and therefore will not be described in detail. It is understood that, prior to the insertion of the device 20 in an intervertebral space between the adjacent vertebrae V5 and V6, an artificial disc, in the form of a PRODISC® artificial disc or one of the artificial disc embodiments disclosed in U.S. Pat. No. 5,314,477, the disclosure of which is incorporated by reference, is removed during revision surgery, thereby defining endplate contours V5a and V6a of the vertebra V5 and V6, respectively, and defining channels V5b and V5c in the vertebra V5 and channels V6b and V6c in the vertebra V6.

Upon insertion of the device 20 in the intervertebral space between the vertebrae V5 and V6, the anchoring flaps 20c, 20d, 20e and 20f extend into the channels V5b, V5c, V6b and V6c, respectively. Moreover, the portions 20a and 20b engage the endplate contours V5a and V6a, respectively. In view of the foregoing engagements, it is understood that the general correspondence between the geometry of the device 20 and the PRODISC® artificial disc or one of the artificial disc embodiments disclosed in U.S. Pat. No. 5,314,477, the disclosure of which is incorporated by reference, facilitates the engagements between the device 20 and the vertebrae V5 and V6, thereby assisting to minimize the need for material removal from the vertebrae V5 and/or V6.

An intervertebral fusion device adapted to be inserted in an intervertebral space between adjacent first and second vertebrae for fusing the first and second vertebrae has been described that includes a first portion adapted to engage the first vertebra, the first portion defining a first geometry wherein at least a portion of the first geometry generally corresponds to at least one portion of a geometry defined by an artificial disc.

A method for fusing adjacent vertebrae has been described that includes inserting an intervertebral fusion device in an intervertebral space between the adjacent vertebrae and engaging a first portion of the intervertebral fusion device with one of the adjacent vertebrae wherein the first portion defines a first geometry that generally corresponds to at least one portion of a geometry defined by an artificial disc.

An intervertebral fusion device has been described that is adapted to be inserted in an intervertebral space between adjacent vertebrae to fuse the adjacent vertebrae and that defines a geometry that generally corresponds to the geometry of an artificial disc adapted to be inserted in the intervertebral space, wherein the device is adapted to be inserted in the intervertebral space and engaged with the adjacent vertebrae after the artificial disc is inserted and removed from the intervertebral space, and wherein the general correspondence between the geometry of the device and the geometry of the artificial disc facilitates the engagements between the device and each of the adjacent vertebrae.

It is understood that one or more portions of, or all of, the device 10, the device 16, the device 18 and/or the device 20 may be modified to have a shape or geometry that generally corresponds to, or matches, the geometry of any one of a wide variety of other artificial disc types, including artificial disc types that are generally known to those skilled in the art such as, for example, a CHARITE® artificial disc or one or more of the artificial disc embodiments disclosed in U.S. Pat. No. 5,401,269, the disclosure of which is incorporated by reference; an ACROFLEX® artificial disc or one or more of the artificial disc embodiments disclosed in U.S. Pat. No. 6,592,624, the disclosure of which is incorporated by reference; a PRESTIGE® ST artificial disc, a PRESTIGE® LP artificial disc, a BRISTOL® artificial disc or one or more of the artificial disc embodiments disclosed in U.S. Pat. Nos. 6,540,785 and 6,113,637, the disclosures of which are incorporated by reference; and one or more of the artificial disc embodiments disclosed in U.S. Pat. Pub. No. 2004/0225366 (U.S. Ser. No. 10/774,157), the disclosure of which is incorporated by reference. In view of the foregoing, it is understood that, in addition to or instead of keel-shaped and/or anchor-flap shaped protrusion portions, any protrusion portions in the devices 10, 16, 18 and/or 20 may be in the form of a wide variety of shapes such as, for example, fins or spikes of varying size.

Moreover, although FIGS. 4 through 6 depict using anterior approaches for the insertion of an intervertebral fusion device between adjacent vertebrae, it is understood that other approaches such as, for example, transverse or posterior approaches, may be used during the insertion of any of the above-described intervertebral fusion devices between adjacent vertebrae.

It is understood that any foregoing spatial references, such as “upper,” “lower,” “above,” “below,” “between,” “vertical,” “angular,” “up,” “down,” “right,” “left,” etc., are for the purpose of illustration only and do not limit the specific orientation or location of the structure described above.

Also, it is understood that each of the above-described embodiments may be combined in whole or in part with one or more of the other above-described embodiments. Moreover, it is understood that one or more of the above-described operational steps of each of the above-described embodiments may be omitted. For example, the devices 10, 16, 18 and 20 may each be inserted in an intervertebral space between adjacent vertebrae without the prior insertion of an artificial disc in the intervertebral space and the subsequent removal of the artificial disc from the intervertebral space. That is, the insertion and removal of the artificial disc prior to the insertion of the device 10, 16, 18 or 20 may be omitted.

It is further understood that each of the above-described embodiments may be combined in whole or in part with other components, devices, systems, methods and/or surgical techniques known to those skilled in the art to provide spinal fusion.

Although exemplary embodiments of this invention have been described in detail above, those skilled in the art will readily appreciate that many other modifications are possible in the exemplary embodiments without materially departing from the novel teachings and advantages of this invention. Accordingly, all such modifications are intended to be included within the scope of this invention as defined in the following claims. In the claims, any means-plus-function clauses are intended to cover the structures described herein as performing the recited function and not only structural equivalents, but also equivalent structures.

What is claimed is:

1. An intervertebral fusion device adapted to be inserted in an intervertebral space between adjacent first and second vertebrae for fusing the first and second vertebrae, the device comprising a first portion adapted to engage the first vertebra, the first portion defining a first geometry wherein at least a portion of the first geometry generally corresponds to at least one portion of a geometry defined by an artificial disc.

2. The device of claim 1 further comprising a second portion adapted to engage the second vertebra, the second portion defining a second geometry wherein at least a portion of the second geometry generally corresponds to at least one other portion of the geometry defined by the artificial disc.

3. The device of claim 2 wherein the general correspondence between the at least a portion of the first geometry and the at least one portion of the geometry defined by the artificial disc facilitates the engagement of the first portion of the device with the first vertebra, and wherein the general correspondence between the at least a portion of the second geometry and the at least one other portion of the geometry defined by the artificial disc facilitates the engagement of the second portion of the device with the second vertebra.

4. The device of claim 3 wherein the artificial disc is adapted to be inserted in the intervertebral space, and wherein the device is adapted to be inserted in the intervertebral space after the artificial disc is inserted and removed from the intervertebral space.

5. The device of claim 4 wherein the device is at least partially composed of an osteogenic material to promote the fusion of the first and second vertebrae.

6. The device of claim 5 wherein the device is monolithic.

7. The device of claim 4 wherein the device further comprises a cage portion extending between the first and second portions of the device.

8. The device of claim 7 wherein the cage portion defines a chamber for receiving a material that is at least partially osteogenic.

9. The device of claim 4 wherein the first and second portions of the device define a dimension generally corresponding to an effective height of the artificial disc.

10. The device of claim 4 wherein the first portion comprises a plate-shaped portion and at least one protrusion portion extending therefrom and adapted to extend in a first channel formed in the first vertebra to further facilitate the engagement between the device and the first vertebra.

11. The device of claim 10 wherein the second portion comprises a plate-shaped portion and at least one protrusion extending therefrom and adapted to extend in a second channel formed in the second vertebra to further facilitate the engagement between the device and the second vertebra.

12. A method for fusing adjacent vertebrae, the method comprising inserting an intervertebral fusion device in an intervertebral space between the adjacent vertebrae and engaging a first portion of the intervertebral fusion device with one of the adjacent vertebrae wherein the first portion defines a first geometry that generally corresponds to at least one portion of a geometry defined by an artificial disc.

13. The method of claim 12 further comprising engaging a second portion of the intervertebral fusion device with the other of the adjacent vertebrae wherein the second portion defines a second geometry that generally corresponds to at least one other portion of the geometry defined by the artificial disc.

14. The method of claim 13 further comprising removing the artificial disc from the intervertebral space between the adjacent vertebrae before the step of inserting.

15. The method of claim 14 further comprising disposing a filler material in the vicinity of the adjacent vertebrae.

16. The method of claim 14 wherein the intervertebral fusion device is at least partially composed of an osteogenic material.

17. The method of claim 14 further comprising disposing an osteogenic material in a chamber formed in the intervertebral fusion device.

18. An intervertebral fusion device adapted to be inserted in an intervertebral space between adjacent vertebrae to fuse the adjacent vertebrae and defining a geometry generally corresponding to the geometry of an artificial disc adapted to be inserted in the intervertebral space, wherein the device is adapted to be inserted in the intervertebral space and engaged with the adjacent vertebrae after the artificial disc is inserted and removed from the intervertebral space, and wherein the general correspondence between the geometry of the device and the geometry of the artificial disc facilitates the engagements between the device and each of the adjacent vertebrae.

19. The device of claim 18 wherein the device is at least partially composed of an osteogenic material to promote the fusion of the adjacent vertebrae.

20. The device of claim 19 wherein the device is monolithic.
